(19) United States
(12) Patent Application Publication
(10) Pub. No.: US 2021/0074399 A1
(43) Pub. Date: Mar. 11, 2021

(54) CLINICAL DECISION SUPPORT SYSTEM WITH TEMPORAL CONTEXT

(71) Applicant: KONINKLIJKE PHILIPS N.V., EINDHOVEN (NL)

(72) Inventors: Eric Cohen-Solal, Monroe, ME (US); Karina Tulipano, Englewood, NJ (US); Victor P.M. Vloemans, Son en Breugel (NL); Roland Opfer, Hamburg (DE); Ingwer Carlsen, Hamburg (DE)

(21) Appl. No.: 17/100,066

(22) Filed: Nov. 20, 2020

Related U.S. Application Data
(63) Continuation of application No. 13/580,387, filed on Oct. 23, 2012.
(60) Provisional application No. 61/310,307, filed on Mar. 4, 2010.

(51) Int. Cl. G16H 15/00

(52) U.S. Cl. CPC G16H 15/00

(57) ABSTRACT

A clinical decision support system (CDSS) includes a CDSS module comprising a digital processing device configured to store patient data and a clinical guideline. The clinical guideline comprises connected nodes representing clinical workflow events, actions, or decisions, wherein contiguous groups of the nodes are grouped into care phases. The CDSS module is configured to associate patient data with patient care phases selected from the care phases of the clinical guideline, and determine patient care phase time intervals for the patient care phases based on acquisition date information for the patient data associated with the patient care phases. The selected patient care phase may be a care phase associated with a clinical guideline node associated with the new patient data, or may be a patient care phase that is consistent with the acquisition date for the new patient data.

### CROSS-REFERENCE TO RELATED APPLICATIONS

This application is a continuation of U.S. patent application Ser. No. 13/580,387, filed on Oct. 23, 2012, which is the U.S. National Phase Application under 35 U.S.C. § 371 of International Application No. PCT/IB2011/050452, filed on Feb. 2, 2011, which claims the benefit of U.S. Provisional Application No. 61/310,307, filed on Mar. 4, 2010, the disclosures of which are hereby incorporated by reference in their entirety herein.

### DESCRIPTION

The following relates to the medical arts, clinical arts, medical diagnostic arts, patient care arts, and related arts.

A clinical decision support system (CDSS) is an interactive electronic system that provides guidance for a physician or other medical personnel in diagnosing and treating a patient. One type of CDSS is defined by a clinical guideline that can be viewed as a directed graph of nodes, with each node representing an action to be taken (such as a medical test or treatment to perform) or a decision to be made. Decision nodes are typically branching points in the directed graph, with the branch to be followed subsequent to the decision node depending upon the decision made at the decision node. The clinical guideline can be highly specific (directed to a specific medical condition) or more general (encompassing a range of medical conditions through the use of suitable decision nodes). A more general clinical guideline is advantageously useful for a wider range of medical conditions. Additionally, a more general clinical guideline encompasses more diverse medical conditions and hence is less likely to “miss” a diagnosis or decision because it lies outside the scope of the clinical guideline.

However, the size of the clinical guideline, as measured by the number of nodes, number of decision points, or other metrics, tends to increase substantially as the scope of the clinical guideline is enlarged. Some clinical guidelines can include dozens, hundreds, or more nodes and decision points. Moreover, in many clinical guidelines similar nodes are repeated, sometimes multiple times. For example, an oncology patient may undergo radiation therapy entailing several radiation therapy sessions, followed by diagnostic testing to assess efficacy of the radiation therapy, followed by further radiation therapy entailing additional radiation therapy sessions, followed by further diagnostic testing, and so forth. Although the repetitions may involve similar nodes, the clinical significance of the treatment and/or patient data may be very different for the different repetitions. By way of example, the aforementioned first radiation therapy may be therapeutic, that is, intended to remove the cancer; whereas the second radiation therapy may be precautionary and intended to prevent recurrence of the cancer. In such a case, a positive test result indicating the presence of cancer during the first (therapeutic) radiation therapy may be “normal”, whereas the same test result indicating the presence of cancer during the second (precautionary) radiation therapy may indicate an unexpected recurrence of the cancer.

The clinical guideline follows a timeline in the sense that the nodes form a directed graph mapping the patient's progress in time. However, the time interval between nodes is not fixed. For example, different oncology patients may remain at a given cancer stage for differing periods. Moreover, patient data is not necessarily acquired in any particular order, with various tests scheduled based on availability of equipment and personnel, based on the judgment of the physician, or other factors. The latency between patient data acquisition and its entry into the CDSS can also vary for between patients, and can even vary for a single patient in the case of repeated patient data acquisitions. These temporal variations in patient data acquisition and entry can be problematic since the significance of patient data can depend critically upon when it was acquired.

The complexity of clinical guidelines and the somewhat ad hoc nature of patient data acquisition and entry into the CDSS can substantially reduce the effectiveness of the CDSS for providing rapid and accurate assessment of a patient's present clinical condition. For example, in the foregoing oncology example, if a positive test indicating cancer was acquired during the first (therapeutic) radiation therapy but not entered into the CDSS until the patient has progressed into the second (precautionary) radiation therapy, then the physician may misinterpret this test result as indicating a problematic recurrence of the cancer. Such misinterpretation remains plausible even if the test result is associated with a node of the therapeutic radiation therapy, since the nodes of the therapeutic and precautionary radiation therapies are similar repetitions.

More generally, a goal of the CDSS is to provide rapid and accurate assessment of the state of the patient and of relevant patient data, so as to assist the physician in making clinical decisions. Existing CDSS approaches would be substantially improved by providing improved techniques for more effectively addressing guideline complexity and the ad hoc nature patient data acquisition and entry.

The following provides new and improved apparatuses and methods which overcome the above-referenced problems and others.

In accordance with one disclosed aspect, an apparatus comprises a clinical decision support system (CDSS) module comprising a digital processing device configured to store patient data and a clinical guideline including connected nodes representing clinical workflow events, actions, or decisions, wherein contiguous groups of the nodes are grouped into care phases. The CDSS module is configured to associate patient data with patient care phases selected from the care phases of the clinical guideline, and to determine patient care phase time intervals for the patient care phases based on acquisition date information for the patient data associated with the patient care phases.

In accordance with another disclosed aspect, a method comprises: storing patient data in a clinical decision support system (CDSS) employing a clinical guideline comprising a directed graph of nodes representing a clinical workflow, wherein contiguous groups of the nodes are grouped into care phases; associating stored patient data with patient care phases selected from the care phases of the clinical guideline; and constructing patient care phase time intervals for the patient care phases such that (i) each patient care phase time interval includes the acquisition time or acquisition times of all patient data associated with the patient care phase and (ii) no point in time is included in more than two patient care phase time intervals. In some embodiments the method is performed by a digital processing device.

In accordance with another disclosed aspect, a storage medium stores: a clinical guideline comprising a directed graph of nodes representing a clinical workflow, wherein contiguous groups of the nodes are grouped into care phases; and instructions executable on a digital processing device to perform a method including receiving patient data associated with patient care performed in accordance with the clinical guideline including receiving an acquisition date for the patient data, associating the patient data with a patient care phase selected from the care phases of the clinical guideline, and creating or updating a patient care phase time interval for the patient care phase wherein the created or updated patient care phase time interval includes the acquisition date.

One advantage resides in a CDSS that provides a physician with a more rapid and accurate assessment of patient condition and the significance of patient data.

Another advantage resides in providing this more rapid and accurate assessment without concomitant substantial increase in the amount of information provided to the CDSS by a physician or other data entry person.

Another advantage resides in reducing a likelihood of error in the assessment of patient condition or the significance of patient data when using a CDSS.

Further advantages will be apparent to those of ordinary skill in the art upon reading and understanding the following detailed description.

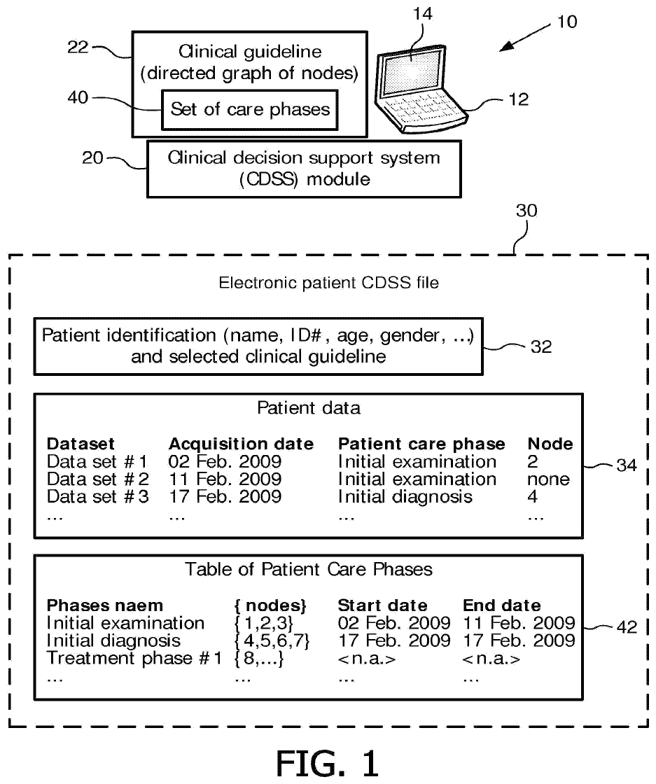

FIG. 1 diagrammatically illustrates a clinical decision support system (CDSS).

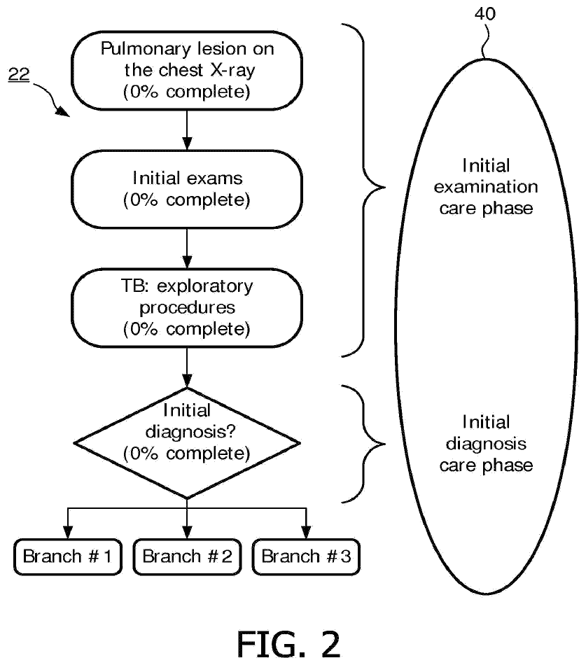

FIG. 2 diagrammatically illustrates a portion of a clinical guideline suitably implemented by the CDSS of FIG. 1, including care phase information.

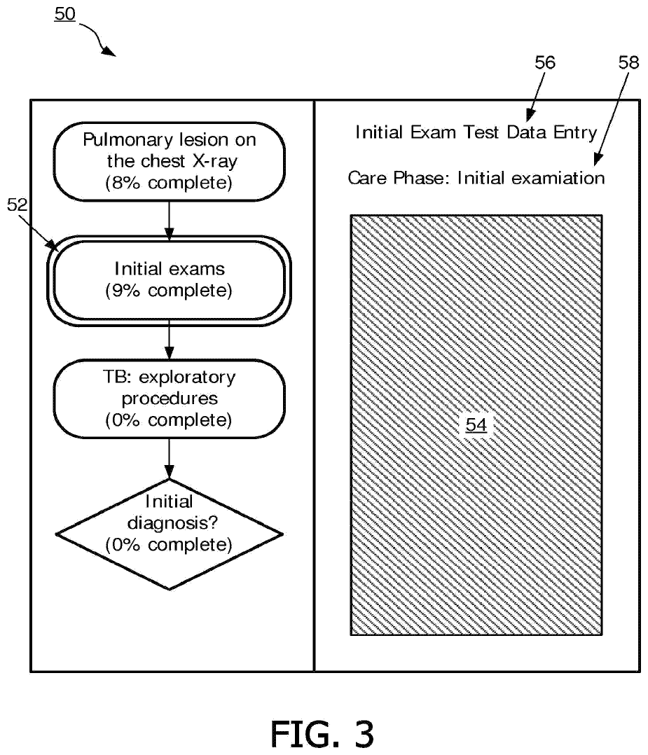

FIG. 3 diagrammatically illustrates a user input dialog window of the CDSS of FIG. 1 by which a physician or other data entry person can input patient data that is associated with a node of the clinical guideline.

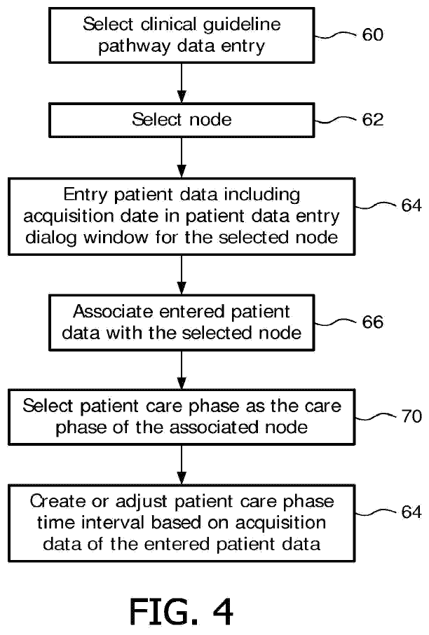

FIG. 4 flowcharts patient data entry using the user input dialog window of FIG. 3, including assignment of a care phase to the patient data.

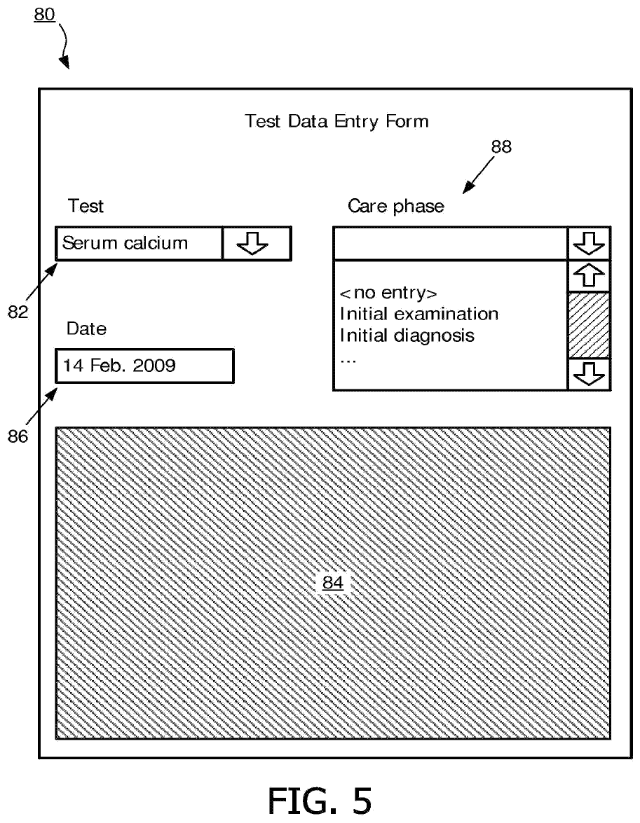

FIG. 5 diagrammatically illustrates a user input dialog window of the CDSS of FIG. 1 by which a physician or other data entry person can input patient data that is not associated with a node of the clinical guideline.

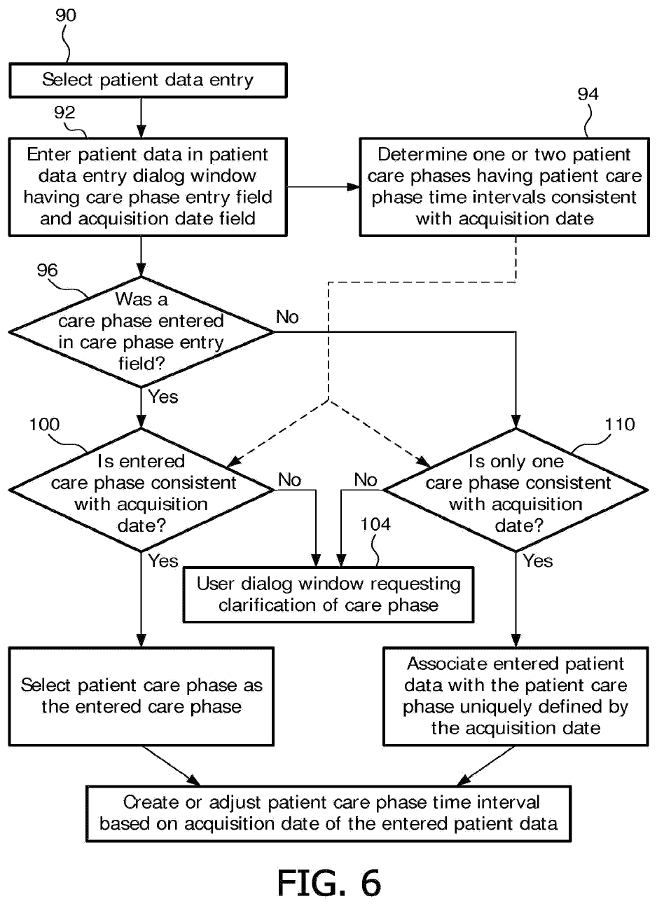

FIG. 6 flowcharts patient data entry using the user input dialog window of FIG. 5, including assignment of a care phase to the patient data.

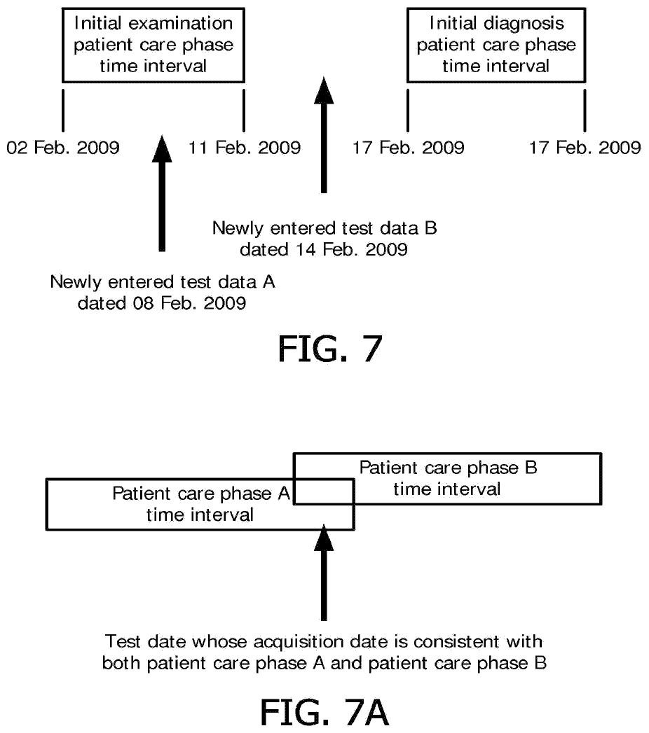

FIG. 7 diagrammatically illustrates assignment of one or two care phases that are consistent with the acquisition date of patient data.

FIG. 7A diagrammatically illustrates assignment of two care phases that are consistent with the acquisition date of patient data, in a variant embodiment in which two care phases overlap in time.

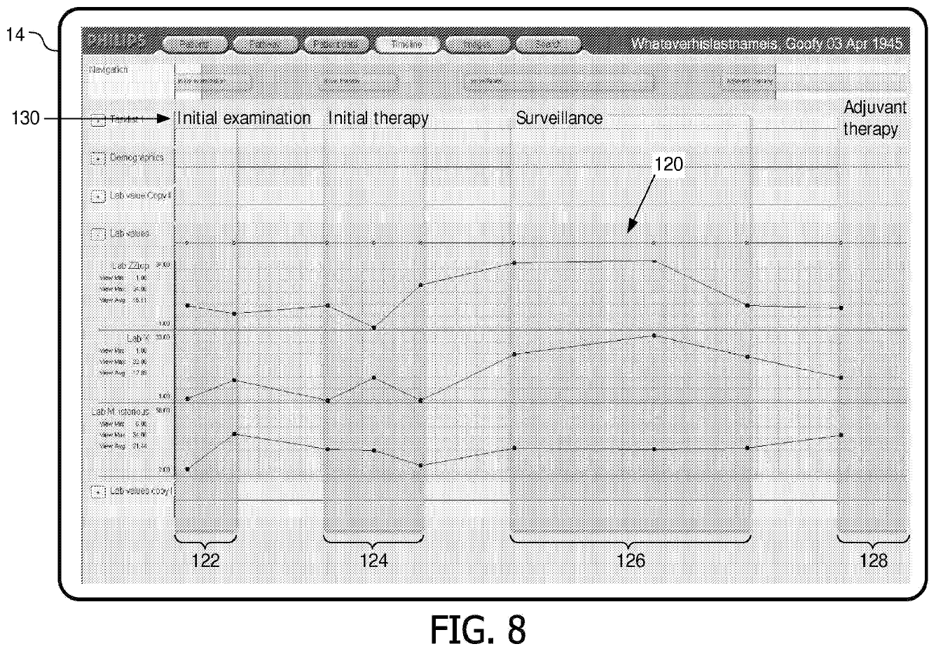

FIG. 8 diagrammatically illustrates a display window generated by the CDSS of FIG. 1 showing a patient's progress through a clinical guideline, including delineation of the various care phases.

With reference to FIG. 1, an illustrative example clinical decision support system (CDSS) is suitably implemented on a computer 10, or on a network server or other digital processing device. The illustrated computer 10 includes an illustrated keyboard 12, and/or a mouse, trackball, or other user input device(s) and an illustrated display 14, and/or a printer, voice synthesizer, or other output device. In the case of other digital processing devices, these or other suitable user input and output devices are suitably provided (for example, a network server may be operatively connected with a remote terminal providing user input/output capability). The digital processing device implementing the CDSS may optionally include multiple cores, parallel processors, multiple computers with a wired and/or wireless network, a graphical processing unit (GPU), analog data processing circuits, or other data processing features or enhancements.

With continuing reference to FIG. 1 and with brief further reference to FIG. 2, the digital processing device 10 implements a CDSS module 20 that operates in conjunction with a clinical guideline 22, which is a directed graph of nodes with each node representing an action to be taken (such as a medical test or treatment to perform) or a decision to be made, and the direction of the graph indicating the progression in time of patient treatment performed in accordance with the clinical guideline. In an illustrative example of an embodiment of the clinical guideline 22 shown in FIG. 2, the nodes include a “Pulmonary Lesion on the Chest X-ray” node, which leads into an “Initial Exams” node, which leads into a “TB: Exploratory procedures” node, which leads into an “Initial diagnosis?” decision node. At the decision node, human input is required to decide the future path of the patient's progress through the clinical guideline. In the example of FIG. 2, the physician or other human user makes a decision at the “Initial diagnosis?” decision node to follow one of three available branches: a “Branch #1” node; a “Branch #2” node; or a “Branch #3” node. In this fashion, the clinical guideline 22 can provide guidance including: identification of relevant tests or other patient data acquisitions to perform, identification of points at which clinical decisions should be made; identification of the available options at a given decision point; and so forth.

The clinical guideline can be displayed on the display 14 in a suitable format, such as that shown in FIG. 2, and the display can include selected information associated with each node. For example, in FIG. 2 nodes include a “percent complete” indicator indicating the extent to which the task at each node has been completed. In illustrative FIG. 2, the clinical guideline is displayed with the “time” direction, that is, the direction of the directed graph, going downward; however, other arrangements can be used, such as having the “time” direction correspond to moving to the right, or even a combination such as progressing forward in time by following edges either to the right or downward from node-to-node.

With continuing reference to FIG. 1, the clinical guideline 22 is suitably stored on a magnetic disk, optical disk, server data storage, read-only memory (ROM), FLASH memory, random access memory (RAM), or other storage medium included in or accessible by the digital processing device 10. The clinical guideline 22 can be generated in various ways. In a typical approach, a physician, team of physicians, or other medically-qualified personnel construct the clinical guideline 22 using suitable design software (not shown) by which these qualified personnel can define nodes and directed edges between nodes so as to map out the various clinical actions (e.g., tests, therapies, or so forth), clinical decisions, and other aspects of treatment of the medical condition or conditions falling within the scope of the clinical guideline 22. Optionally, these qualified personnel can also associate various information with the nodes, such as: textual or graphical instructions for performing the actions, tests, or so forth associated with the nodes; advice for assisting in making clinical decisions associated with decision nodes; value ranges for patient test results that are considered to be “normal”; or so forth.

The CDSS module 20 of FIG. 1 maintains an electronic patient CDSS file 30 for each patient. By way of illustration, the single electronic patient CDSS file 30 for a single patient is shown in FIG. 1 as an illustrative example; however, in general the CDSS maintains an electronic patient CDSS file for each patient. For example, if the CDSS is used to provide clinical guidance for treatment of 125 different patients, then the CDSS suitably maintains 125 electronic patient CDSS files, one for each patient. Moreover, it is to be understood that the electronic patient CDSS file 30 for a single patient may optionally be organized into multiple files or other logical components for the purpose of storage and manipulation by the CDSS module 20.

The electronic patient CDSS file 30 stores information pertaining to the patient. In the illustrative embodiment, this information includes a patient identification component 32 storing information including: patient name; patient identification (ID) number; patient characteristics such as gender, age, or so forth; and the clinical guideline being employed for the patient. In regard to this last illustrative item, although FIG. 1 illustrates the single clinicial guideline 22 it is to be understood that in some embodiments there may be plural available clinical guidelines, and the physician can select the clinical guideline to use with a particular patient. By way of example, a patient apparently suffering from throat cancer may be treated using a throat cancer clinical guideline, a patient apparently suffering from lung cancer may be treated using a lung cancer clinical guideline, and so forth.

The electronic patient CDSS file 30 also includes a patient data component 34 that stores patient data such as medical test results, medical images (e.g., MR or CT images), textual annotations or notes entered by the patient's physician or other medical personnel, or so forth. In the illustrative embodiment, the patient data is organized by data set, and each data set is tagged by its acquisition date, and is optionally also associated with a node of the clinical guideline 22. By way of example: a stored “Data Set #1” is tagged with acquisition date 2 Feb. 2009 and is associated with node “2”; a stored “Data Set #2” is tagged with acquisition date 11 Feb. 2009 and is not associated with any node of the clinical guideline; a stored “Data Set #3” is tagged with acquisition date 17 Feb. 2009 and is associated with node “4”; and so forth.

A goal of the CDSS is to provide rapid and accurate assessment of the state of the patient and of relevant patient data, so as to assist the physician in making clinical decisions. Toward this end, in the illustrative CDSS of FIG. 1 contiguous groups of nodes are grouped into a set of care phases 40. Each care phase is a contiguous group of connected nodes of the clinical guideline 22. Because the clinical guideline 22 has no time scale, the care phases 40 do not have a temporal aspect or metric. However, since the contiguous group of connected nodes define contiguous sequence of actions, events, or decisions that occur in the sequence defined by the directed graph, a care phase defined by a contiguous group of connected nodes corresponds to a contiguous (albeit unspecified) time interval. The set of care phases 40 are preferably chosen to provide clinically useful and meaningful groupings of information so as to facilitate rapid and accurate assessment of the state of the patient and of relevant patient data. In general, a care phase is defined as a group of nodes that are connected (and hence temporally contiguous) in the clinical guideline 22. This is illustrated in FIG. 2, where the first three nodes are grouped together as an “initial examination” care phase, while the next decision node forms a group of one node comprising the “initial diagnosis” care phase. As illustrated by this latter example, the contiguous group of connected nodes defining a care phase can include as few as a single node of the clinical guideline 22.

The set of care phases 40 are used to group the patient data into patient care phases selected from the care phases 40 of the clinical guideline 22. Because the patient care phases are associated with a patient whose treatment occurs in time, the patient care phases can have associated patient care phase time intervals. Each patient care phase time interval is a continuous time interval that includes the acquisition dates of all patient data associated with that patient care phase. In general, there may or may not be a time gap between two neighboring or temporally adjacent patient care phases. In some embodiments, patient care phase time intervals do not overlap in time. In other embodiments, two neighboring or temporally adjacent patient care phase time intervals may overlap in time. Typically, it is desirable to have either no overlapping patient care phase time intervals (as in some embodiments) or at most two neighboring or temporally adjacent patient care phase time intervals may overlap in time (as in some other embodiments).

With continuing reference to FIG. 1, each data set of the patient data 34 is associated with a patient care phase that is selected from the set of care phases 40 of the clinical guideline 22. In the illustrative example, “Data Set #1” acquired 2 Feb. 2009 and “Data Set #2” acquired 11 Feb. 2009 are both associated with the “Initial examination” patient care phase, while “Data Set #3” acquired 17 Feb. 2009 is associated with the “Initial diagnosis” patient care phase. A table of patient care phases 42 is also stored as part of the electronic patient CDSS file 30. The table of patient care phases 42 lists the patient care phases and their corresponding patient care phase time intervals. Patient data associated with the “Initial examination” patient care phase were acquired on 2 Feb. 2009 and on 11 Feb. 2009. Since the “Initial examination” patient care phase time interval must be contiguous, it follows that the “Initial examination” patient care phase time interval must at least include the interval [2 Feb. 2009, 11 Feb. 2009], where the hard brackets [. . . ] denote inclusion of the end points in the time interval. The “Initial diagnosis” patient care phase is associated only with a single data set having a single acquisition date, namely 17 Feb. 2009. Accordingly, the “Initial diagnosis” patient care phase time interval must include at least the interval [17 Feb. 2009, 17 Feb. 2009]. These time intervals are shown in the table of patient care phases 42 of FIG. 1.

Assignment of patient data to a patient care phase contextualizes the patient data. If the patient data is not associated with a particular node of the clinical guideline 22, then the assigned care phase provides contextualization for the patient data. Even if the patient data is associated with a particular node of the clinical guideline 22, the additional ssignment of the patient data to a particular care phase can provide further contextualization that may benefit the physician if, for example, the associated node is similar to other nodes that are repeated at different points in the clinical guideline 22.

When new patient data are acquired, the new patient data are associated with a patient care phase and the corresponding patient care phase time interval is created or updated, if needed, such that the patient care phase time interval includes the acquisition date of the new patient data.

With reference to FIG. 3, in one approach a dialog window 50 is used to input patient data that is associated with a node of the clinical guideline 22. In a lefthand side of the illustrative dialog window 50, an associated node 52 is highlighted in a graphical representation of a portion of the clinical guideline 22. The highlighting can utilize any distinctive visual effect, such as color, boldface outlining (as illustrated), flashing, or so forth. The associated node in the illustrative example is the “Initial exams” node 52. In a righthand side of the illustrative dialog window 50, a user input region 54 is provided in which the user can input patient data associated with the associated node 52. The input region 54 is shown diagrammatically as a box in FIG. 3; however, since the associated node 52 is known the input region 54 is optionally formatted specifically for input of a type of patient data expected to be associated with the node 52. Additionally, the righthand side of the illustrative dialog window 50 is identified by a node identification annotation 56 and by a care phase annotation 58, the latter being the care phase into which the associated node 52 is grouped.

With reference to FIG. 4, the processing of the new patient data received via the illustrative dialog window 50 of FIG. 3 is described. In an operation 60, the user selects the clinical guideline pathway data entry, and in an operation 62 the user selects the associated node 52. In some embodiments, the operations 60, 62 are simultaneously accomplished by double-clicking on the associated node 52. In some embodiments, the operation 62 is performed first by right-clicking on the associated node 52 to bring up a context-sensitive menu and the operation 60 is then performed by selecting a data input option from the context-sensitive menu, or so forth. Other approaches are also contemplated. The operations 60, 62 generate the dialog window 50 of FIG. 3, and the user inputs patient data including an acquisition date in the dialog window 50 of FIG. 3 in an operation 64. In an operation 66, the entered (i.e., new) patient data are associated with the associated node 52. This is straightforward since the associated node 52 was selected in operation 62 in order to generate the dialog window 50. In an operation 70, the patient care phase for the entered (i.e., new) patient data is selected as a care phase into which the associated node 52 is grouped in the set of care phases 40. In embodiments in which each node is grouped into no more than one care phase, the operation 70 is straightforward and unambiguous. In embodiments in which a node may be grouped into two overlapping care phases, the operation 70 may suitably entail bringing up another dialog box (not shown) asking the user which of the two different care phases into which the associated node 52 is grouped should be assigned to the new patient data.

In an operation 72, the patient care phase time interval of the patient care phase selected in the operation 70 is created or adjusted, if needed, to ensure that the patient care phase time interval includes the acquisition date of the entered (i.e., new) patient data. If this is the first instance in which patient data is associated with the patient care phase selected in the operation 70, then the operation 72 creates the corresponding patient care phase time interval and suitably assigns it the value [t_{new}, t_{new}] where the time trim is the acquisition date associated with the entered (i.e., new) patient data. If this is not the first instance of patient data associated with the patient care phase (that is, if the patient care phase and a corresponding patient care phase time interval already exists), then the operation 72 adjusts the patient care phase time interval (if needed) to ensure that it includes the acquisition date of the new patient data. For example, if the current (unadjusted) patient care phase time interval is [t₁, t₂] and the acquisition date t_{new}<t₁ then the adjusted patient care phase time interval is suitably [t_{new}, t₂]. Similarly, if the current (unadjusted) patient care phase time interval is [t₁, t₂] and the acquisition date t_{new}>t₂ then the adjusted patient care phase time interval is suitably [t₁, t_{new}].

The approach of FIGS. 3 and 4 assumes that there is an associated node corresponding to the new patient data. However, in some instances the patient data are not associated with any particular node (e.g., see “Data Set #2” in the patient data 34 of FIG. 1), or the physician or other data entry person may not know the associated node or want to take the time to identify the associated node. In such instances, it is advantageous to provide a mechanism by which the data entry person can enter new patient data without specifying any associated node.

With reference to FIG. 5, such a data entry dialog window 80 is illustrated. The dialog window 80 includes a data type (e.g., test) selection input 82 which may, for example, provide a drop-down list of available data types. In the illustrative example, the user has selected a “Serum calcium” test data entry. A data entry region 84 is diagrammatically depicted in FIG. 5 by a box. In general, the data entry region 84 is suitably formatted for the data type selected by the user via the selection input 82. For example, the selection of the serum calcium test input via selection input 82 (as illustrated) may cause the data entry region 84 to comprise a single digital data input for receiving a quantitative serum calcium value. On the other hand, if the user had selected an imaging data type via the selection input 82, the data entry region 84 might suitably include (i) a file input dialog via which the user could input or select a data file containing the image, and (ii) an image preview sub-window in which the selected image is displayed. There are merely examples. The dialog window 80 also includes an acquisition date input 86 via which the user inputs an acquisition date for the new patient data. The dialog window 80 still further includes a care phase selection input 88 via which the user optionally selects one of the care phases 40 to associate to the new input patient data. In the illustrative example the care phase selection input 88 is a drop-down list input listing the care phases 40 and also providing a default value of <no entry>. The user may choose to use the care phase selection input 88 to affirmatively select one of the care phases 40 of the clinical guideline 22 as the patient care phase to be associated with the input patient data. Alternatively, if the user goes with the default <no entry>, then the acquisition date provided via the input 86 is used to choose the patient care phase.

With reference to FIG. 6, the processing of the new patient data received via the illustrative dialog window 80 of FIG. 5 is described. In an operation 90 the user selects the dialog window 80, and in an operation 92 the user fills in the entries 82, 84, 86 and optionally care phase input 88.

With continuing reference to FIG. 6 and with brief reference to FIGS. 7 and 7A, in an operation 94 the CDSS module 20 determines one or two patient care phases that are consistent with the acquisition date for the new patient data entered via the acquisition date input 86 (see FIG. 5). The table of patient care phases 42 (see FIG. 1) stores the start and end time of each patient care phase so as to define the patient care phase time interval for each patient care phase. In the embodiment of FIG. 7, the CDSS module 20 is configured to ensure that no two patient care phases overlap in time. If the acquisition date is included in a patient care phase time interval, the then the acquisition date is consistent with only one patient care phase, namely the patient care phase whose patient care phase time interval includes the acquisition date. An example of this situation is the 8 Feb. 2009 acquisition date for “Test Data A” shown in FIG. 7. The 8 Feb. 2009 date is included in the “Initial examination” patient care phase time interval, and accordingly the 8 Feb. 2009 acquisition date is consistent with only the “Initial examination” patient care phase.

If the acquisition date lies between two neighboring patient care phase time intervals, then the acquisition date is consistent with either of these two patient care phases. An example of this situation is the 14 Feb. 2009 acquisition date for “Test Data B” shown in FIG. 7. The 14 Feb. 2009 date lies between the “Initial examination” patient care phase time interval and the “Initial diagnosis” patient care phase time interval, and accordingly the 14 Feb. 2009 acquisition date is consistent with two patient care phases, namely the “Initial examination” patient care phase and the “Initial diagnosis” patient care phase. With brief reference to FIG. 7A, in embodiments in which two patient care phase time intervals may overlap, there are still one or at most two patient care phases that are consistent with a given patient data acquisition date.

With continuing reference to FIG. 6, at a decision point 96 it is determined whether or not the user input a care phase via the care phase selection input 88. If yes, then an operation 100 checks to verify that the acquisition date for the new patient data (as determined in the operation 94) is consistent with the user-entered care phase. If the input care phase is determined in the operation 100 to be consistent with the acquisition date, then in an operation 102 the new patient data are associated with the user-input care phase. On the other hand, if the input care phase is determined in the operation 100 to be not consistent with the acquisition date, then a remedial operation 104 is invoked. In the illustrated embodiment, the remedial operation 104 includes providing a user dialog window explaining the discrepency and requesting user input (for example, corrected user input of the acquisition date and/or care phase) to resolve the discrepancy.

On the other hand, if at the decision point 96 it is determined that no user-input care phase was provided via the care phase selection input 88, then a decision block 110 uses the output of the operation 94 to attempt to assign a uniquely identified patient care phase based on the acquisition date. The operation 94 determines one or two patient care phases that are consistent with the acquisition date. At the decision block 110, if only one patient care phase was found in operation 94 to be consistent with the acquisition date, then decision block 110 transfers flow to an operation 112 where the new patient data are associated with the single patient care phase that is consistent with the acquisition date. On the other hand, if two patient care phases were found in operation 94 to be consistent with the acquisition date, then the remedial operation 104 is invoked, which again in the illustrated embodiment includes providing a user dialog window explaining the discrepency and requesting user input (for example, a corrected user input of the acquisition date and/or entry of a care phase) to resolve the discrepancy.

After the patient data are assigned a patient care phase through either operation 102 or operation 112, an operation 114 is invoked to create or adjust the patient care phase time interval of the patient care phase to which the new patient data are assigned. In some instances, the operation 114 does nothing—for example, in FIG. 7 the assignment of “Test Data A” to the “Initial examination” patient care phase does not entail any creation or adjustment of the “Initial examination” patient care phase time interval because the acquisition date of 8 Feb. 2009 is already included in the “Initial examination” patient care phase time interval. On the other hand, if “Test Data B” is assigned to the “Initial examination” patient care phase then the end date of the “Initial examination” patient care phase time interval is suitably increased to include the 14 Feb. 2009 acquisition date of “Test Data B”; in other words, the the “Initial examination” patient care phase time interval is increased from [2 Feb. 2009, 11 Feb. 2009] to [2 Feb. 2009, 14 Feb. 2009]. If the new patient data is the first data assigned to a patient care phase, such that the patient care phase is created to correspond to the new patient data, then the newly created patient care phase is suitably given the patient care phase time interval [d_{A}, d_{A}], where d_{A} is the acquisition date of the new patient data.

With reference to FIG. 8, the patient care phases are used to improve readability of a display 120 of at least some of the stored patient data as a function of time within a selected time interval. The improved readability is provided by, in the displaying, delineating the patient care phase time intervals that are included in or overlap the selected time interval. The illustrative display 120 is a patient data timeline displayed on the display device 14 (see also FIG. 1). In the display 120, four patient care phase time intervals 122, 124, 126, 128 (the last shown in part) are delineated by shading, with textual patient care phase labels 130 shown above the shaded areas. The delineated patient care phase time intervals 122, 124, 126, 128 with labels 130 facilitate rapid and accurate assessment by the physician of the content of the patient data timeline. It will be appreciated that other delineations such as coloration, vertical delineation lines, or so forth may be used.

This application has described one or more preferred embodiments. Modifications and alterations may occur to others upon reading and understanding the preceding detailed description. It is intended that the application be construed as including all such modifications and alterations insofar as they come within the scope of the appended claims or the equivalents thereof.

Having thus described the preferred embodiments, the invention is now claimed to be:

1. A method comprising:
representing, on a display in communication with a digital processing device, a clinical guideline for a medical treatment of a patient as a directed graph of nodes, wherein each node in the directed graph represents an action to be taken or decision to be made during the medical treatment, wherein the direction of the graph indicates a time progression of the medical treatment, wherein different sets of one or more contiguous nodes of the directed graph are grouped on the display into different care phases, and wherein a visual representation of a time interval of at least one of the care phases is enhanced on the display by delineating a start and an end of the time interval;
receiving, via a user input device in communication with the display, user input for associating a patient data set having an acquisition date with a selected node of the directed graph, a selected care phase of the different care phases, or both; and
updating, based on the user input, the visual representation of one or more time intervals of the directed graph by adjusting a delineation of the start and end of one or more time intervals such that the acquisition date of the patient data set is within the delineation of the time interval of the care phase selected by the user input or associated with the selected node.

2. The method of claim 1 further comprising displaying an input region configured for receiving the patient data set, the acquisition date of the patient data set, or both.

3. The method of claim 2, wherein the input region is displayed in response to selection of a node of the directed graph.

4. The method of claim 3, wherein the displayed input region is formatted specifically for input of a type of patient data expected to be associated with the selected node.

5. The method of claim 3 further comprising highlighting the selected node of the directed graph on the display.

6. The method of claim 4, further comprising displaying, if the selected node is associated with a plurality of care phases, a dialog box for selecting a care phase from the a plurality of care phases with which the patient data set is to be associated.

7. The method of claim 1, wherein said receiving user input comprises displaying a dialog window comprising a first input element for selecting a data type of the patient data set and a second input element for specifying the acquisition date of the patient data set, the method further comprising associating the patient data set with one of the different care phases based, at least in part, on the acquisition date specified via the second input element.

8. The method of claim 7, wherein the dialog window further comprises a third input element for selecting a care phase from a subset of the different care phases, the method further comprising associating the patient data set with the selected care phase and updating the visual representation of the time interval of the selected care phase to include the acquisition date of the patient data set within the delineation of the start and an end of the time interval of the selected care phase.

9. The method of claim 1, wherein the clinical guideline comprises a plurality of paths, and wherein the method further comprises selecting, by the digital processing device, one of the plurality of paths responsive to user input at a decision node of the directed graph, and updating the directed graph to follow the selected path of the clinical guideline.

10. The method of claim 1 further comprising displaying a percent complete indicator for at least one of the nodes of the directed graph.

11. The method of claim 1, wherein the digital processing device is configured to set the time intervals of the selected care phases into which nodes are grouped such that none of the time intervals overlap at any point in time.

12. A non-transitory storage medium comprising instructions for providing a user interface on a computer, which instructions when executed by the computer are configured to cause the computer to perform the method of claim 1.

13. A system for clinical decision support in medical treatment, the system comprising a digital processor in communication with a display and a user input device, wherein the system is configured to provide, on the display and responsive to the digital processor, a user interface comprising:
representing, on the display, a first clinical guideline for a medical treatment of a patient as a graph of connected nodes, wherein each node in the graph represents an action to be taken or decision to be made during the medical treatment, wherein a direction of the graph indicates a time progression of the medical treatment, and wherein one or more contiguous nodes of the graph are assigned to at least one care phase on the display, an wherein a visual representation of a time interval of the at least one of the care phase is enhanced on the display by delineating a start and an end of the time interval;
receiving, via the user input device, user input for associating a patient data set havign an acquisition date with a node of the graph, with a care phase of the first clinicla guideline, or with both a node of the graph and a care phase of the firs clinical guidline; and
updating the visual representation of one or more time intervals on the display by adjusting a delineation of the start and end of the one or more time intervals based on the user input.

14. The system of claim 13, wherein the user interface further comprises displaying an input region configured for receiving at least one of the patient data set and the acquisition date of the patient data set to be associated with the node in response to selecting the node on the display.

15. The system of claim 14, wherein the user interface further comprises at least one of:
highlighting the node on the display responsive to selection of the node; and
displaying a percent complete indicator for at least one of the nodes of the graph of connected nodes.

16. The system of claim 13 further comprising a data storage device that stores a plurality of clinical guidelines including the first clinical guideline.

17. The system of claim 16, wherein the data storage device further stores a patient file for at least one patient, the patient file comprising an identification of at least one of the plurality of clinical guidelines and one or more patient data sets, each having a respective acquisition date, and associated with a care phase of the at least one of the plurality of clinical guidelines and a respective node.

18. The system of claim 13, wherein the digital processing device is configured to group nodes of the graph such that the time intervals of no more than two care phases overlap at any given point in time.

19. The system of claim 18, wherein the user interface is configured to display a dialog for receiving the acquisition date of the patient data set and:
if the date is consistent with only a single care phase, assigns the data set based on the acquisition date; and
if the date is consistent with a plurality of care phases, displays a further dialog for receiving user input regarding the selected care phase.

20. The system of claim 13, wherein the clinical guideline comprises a plurality of paths, and wherein the method further comprises selecting, by the digital processing device, one of the plurality of paths responsive to user input at a decision node of the directed graph, and updating the directed graph to follow the selected path of the clinical guideline.
